SEQUENCE LISTING (25 sequences)

SEQ ID NO: 1 - DNA, 415 bp, Artificial Sequence
  misc_feature: cDNA
gggagacccaagcttggtaccgagctcggatccactagtaacggccgccagtgtgctggaattcggcttggaccaagcttaaactcactccaatcccggctgcgtcccggctggacttgtccggctggttcgttgctggatacaacgggggagacatatatcacagcctgtctcgtgcccgaccccgttggtttatgttgtgcctactcctactttctgtaggggtaggcatttacctgctccccaaccgatgaacggggggctaaacactccaggccaataggccatcccctttttttttttttctttccttctttggtggctccatcttagccctagtcacggctagctgtgaaaggtccgtgagccgcatgactgcagagagtgctgatactggcctctctgcagatcatgt

SEQ ID NO: 2 - DNA, 40 bp, Artificial Sequence
  misc_feature: oligonucleotide
tctagacatgatctgcagagaggccagtatcagcactctc

SEQ ID NO: 3 - DNA, 19 bp, Artificial Sequence
  misc_feature: oligonucleotide
atggggcaaaggacgtccg

SEQ ID NO: 4 - DNA, 27 bp, Artificial Sequence
  misc_feature: oligonucleotide
ggaccaagcttaaactcactccaatcc

SEQ ID NO: 5 - DNA, 350 bp, Artificial Sequence
  misc_feature: template 24-1
gggagacccaagcttaaactcactccaatcccggctgcgtcccggctggacttgtccggctggttcgttgctggatacaacgggggagacatatatcacagcctgtctcgtgcccgaccccgttggtttatgttgtgcctactcctactttctgtaggggtaggcatttacctgctccccaaccgatgaacggggggctaaacactccaggccaataggccatcccctttttttttttttctttccttctttggtggctccatcttagccctagtcacggctagctgtgaaaggtccgtgagccgcatgactgcagagagtgctgatactggcctctctgcagatcatgt

SEQ ID NO: 6 - DNA, 425 bp, Artificial Sequence
  misc_feature: template 128-4
gggagacccaagcttaaactcactccaatcccggctgcgtcccggctggacttgtccggctggttcgttgctggatacaacgggggagacatatatcacagcctgtctcgtgcccgaccccgttggtttatgttgtgcctactcctactttctgtaggggtaggcatttacctgctccccaaccgataaacggggagccaaacactccaggccaataggccatccctttttttttttgtttttttttttttttttttttttttttttcttttttttttttttttttttttttttttttccttttccttttttttttatattccttctggtggctccatcttagccctagtcacggctagctgtgaaaggtccgtgagccgcatgactgcagagagtgctgatactggcctctctgcagatcatgt

SEQ ID NO: 7 - DNA, 417 bp, Artificial Sequence
  misc_feature: template 130-21
gggagacccaagcttaaactcactccaatcccggctgcgtcccggctggacttgtccggctggttcgttgctggatacaacgggggagacatatatcacagcctgtctcgtgcccgaccccgttggtttatgttgtgcctactcctactttctgtaggggtaggcatttacctgctccccaaccgataaacggggagccaaacactccaggccaataggccatcccttttttttttttttggtttttttttttttttttttttttttttttttttttttttttttttttcctttttccttttttttttatattccttctggtggctccatcttagccctagtcacggctagctgtgaaaggtccgtgagccgcatgactgcagagagtgctgatactggcctctctgcagatcatgt

SEQ ID NO: 8 - DNA, 422 bp, Artificial Sequence
  misc_feature: template 150-41
gggagacccaagcttaaactcactccaatcccggctgcgtcccggctggacttgtccggctggttcgttgctggatacaacgggggagacatatatcacagcctgtctcgtgcccgaccccgttggtttatgttgtgcctactcctactttctgtaggggtaggcatttacctgctccccaaccgataaacggggagccaaacactccaggccaataggccatccctttttttttttttgttttttttttttttttttttttttttttttttttttttttttttttttttttttttttcctttttttttttttatattccttctggtggctccatcttagccctagtcacggctagctgtgaaaggtccgtgagccgcatgactgcagagagtgctgatactggcctctctgcagatcatgt

SEQ ID NO: 9 - DNA, 18 bp, Artificial Sequence
  misc_feature: oligonucleotide
tccacagttactctccag

SEQ ID NO: 10 - DNA, 24 bp, Artificial Sequence
  misc_feature: oligonucleotide
taggcatttacctgctccccaacc

SEQ ID NO: 11 - DNA, 1884 bp, Artificial Sequence
  misc_feature: HTaA5B polymerase
atgtcgtactaccatcaccatcaccatcacgattacgatatcccaacgaccgaaaacctgtattttcagggcgccatggatccggaattcgaggacgtcgtctgctgctcgatgtcctacacatggacaggcgccctgatcacgccatgcgccgcggaggaaagccagctgcccatcaacgcgttgagcaactctttggtgcgtcatcgcaacatggtctattccacaacatcccgcagcgcggccctgcggcagaagaaggtcacctttgacagactgcaagtcctggacgaccactaccgggacgtgctcaaggagatgaaggcgaaggcgtccacagttaaggccaaactactatcagtagaagaagcctgtaagctgacgcccccacattcggccaaatccaagtttggctatggggcaaaggacgtccggaacctatccagcaaggccgttgaccacatccgctccgtgtggaaggacttgctggaagacactgagacaccaattgacaccaccatcatggcgaaaaatgaggttttctgcgtccaaccagagaaaggaggccgcaagccagctcgccttatcgtattcccagacctgggagttcgtgtatgcgagaaaatggccctctacgacgtggtctccacccttcctcaggccgtgatgggctcctcatacggattccaatactctcccaagcagcgggtcgagttcctggtgaatgcctggaaatcaaagaaatgccctatgggcttctcatatgacacccgctgttttgactcaacggtcactgagagcgacatccgtgttgaggagtcaatctaccaatgttgtgacttggcccccgaagccagacaggctataaagtcgctcacagagcggctctatatcgggggtcccctgaccaattcaaaagggcagaactgcggctatcgccggtgccgcgcgagcggcgtgctgacgaccagctgcggtaataccctcacatgttacttgaaggcctctgcggcctgtcgagctgccaagctccaggactgcacgatgctcgtgaacggagacgaccttgtcgttatctgcgagagcgcgggaacccaggaggatgcggcgaacctacgagtcttcacggaggctatgactaggtactctgccccccccggggacctgccccaaccagaatacgacttggagttgataacatcatgctcctccaatgtgtcggtcgcgcacgatgcatccggcaaaagggtgtactacctcacccgtgaccccaccaccccccttgcgcgggctgcgtgggagacagctagacacactccaatcaactcctggctaggcaatatcatcatgtatgcgcccaccttatgggcaaggatggttctgatgactcatttcttctccatcctcctagcccaggagcaacttgaaaaagccctggattgtcagatctacggggcctgttactccattgagccacttgacctacctcagatcattgaacgactccacggtcttagcgcattttcactccacagttactctccaggtgaaatcaatagggtggcttcatgcctcaggaaacttggggtaccacccttgcgagtctggagacatcgggccagaagtgtccgcgctaagctgctgtcccagggggggagggctgccacttgtggcaagtacctcttcaactgggcagtaaggaccaagcttaaactcactccaatcccggctgcgtcccggctggacttgtccggctggttcgttgctggatacaacgggggagacatatatcacagcctgtctcgtgcccgaccccgttggtttatgttgtgcctactcctactttctgtaggggtaggcatttacctgctccccaaccgatga

SEQ ID NO: 12 - protein, 627 aa, Artificial Sequence
  misc_feature: HTaA5B polymerase
MSYYHHHHHHDYDIPTTENLYFQGAMDPEFEDVVCCSMSYTWTGALITPCAAEESQLPINALSNSLVRHRNMVYSTTSRSAALRQKKVTFDRLQVLDDHYRDVLKEMKAKASTVKAKLLSVEEACKLTPPHSAKSKFGYGAKDVRNLSSKAVDHIRSVWKDLLEDTETPIDTTIMAKNEVFCVQPEKGGRKPARLIVFPDLGVRVCEKMALYDVVSTLPQAVMGSSYGFQYSPKQRVEFLVNAWKSKKCPMGFSYDTRCFDSTVTESDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAANLRVFTEAMTRYSAPPGDLPQPEYDLELITSCSSNVSVAHDASGKRVYYLTRDPTTPLARAAWETARHTPINSWLGNIIMYAPTLWARMVLMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVRTKLKLTPIPAASRLDLSGWFVAGYNGGDIYHSLSRARPRWFMLCLLLLSVGVGIYLLPNR

SEQ ID NO: 13 - DNA, 45 bp, Artificial Sequence
  misc_feature: oligonucleotide
ccaataggccattttttttttttttttttctttccttctttggtg

SEQ ID NO: 14 - DNA, 42 bp, Artificial Sequence
  misc_feature: oligonucleotide
ggaaagaaaaaaaaaaaaaaaaaatggcctattggcctggag

SEQ ID NO: 15 - DNA, 41 bp, Artificial Sequence
  misc_feature: oligonucleotide
cccctttttttttttttctttttttctttggtggctccatc

SEQ ID NO: 16 - DNA, 41 bp, Artificial Sequence
  misc_feature: oligonucleotide
gccaccaaagaaaaaaagaaaaaaaaaaaaaggggatggcc

SEQ ID NO: 17 - DNA, 45 bp, Artificial Sequence
  misc_feature: oligonucleotide
ccaataggccattttttttttttttttttctttttttctttggtg

SEQ ID NO: 18 - DNA, 45 bp, Artificial Sequence
  misc_feature: oligonucleotide
caccaaagaaaaaaagaaaaaaaaaaaaaaaaaatggcctattgg

SEQ ID NO: 19 - DNA, 350 bp, Artificial Sequence
  misc_feature: template 24-1 (m1)
gggagacccaagcttaaactcactccaatcccggctgcgtcccggctggacttgtccggctggttcgttgctggatacaacgggggagacatatatcacagcctgtctcgtgcccgaccccgttggtttatgttgtgcctactcctactttctgtaggggtaggcatttacctgctccccaaccgatgaacggggggctaaacactccaggccaataggccattttttttttttttttttctttccttctttggtggctccatcttagccctagtcacggctagctgtgaaaggtccgtgagccgcatgactgcagagagtgctgatactggcctctctgcagatcatgt

SEQ ID NO: 20 - DNA, 350 bp, Artificial Sequence
  misc_feature: template 24-1 (m2)
gggagacccaagcttaaactcactccaatcccggctgcgtcccggctggacttgtccggctggttcgttgctggatacaacgggggagacatatatcacagcctgtctcgtgcccgaccccgttggtttatgttgtgcctactcctactttctgtaggggtaggcatttacctgctccccaaccgatgaacggggggctaaacactccaggccaataggccatcccctttttttttttttctttttttctttggtggctccatcttagccctagtcacggctagctgtgaaaggtccgtgagccgcatgactgcagagagtgctgatactggcctctctgcagatcatgt

SEQ ID NO: 21 - DNA, 350 bp, Artificial Sequence
  misc_feature: template 24-1 (dm)
gggagacccaagcttaaactcactccaatcccggctgcgtcccggctggacttgtccggctggttcgttgctggatacaacgggggagacatatatcacagcctgtctcgtgcccgaccccgttggtttatgttgtgcctactcctactttctgtaggggtaggcatttacctgctccccaaccgatgaacggggggctaaacactccaggccaataggccattttttttttttttttttctttttttctttggtggctccatcttagccctagtcacggctagctgtgaaaggtccgtgagccgcatgactgcagagagtgctgatactggcctctctgcagatcatgt

SEQ ID NO: 22 - RNA, 25 nt, Artificial Sequence
  misc_feature: RNA initiation site
uuuuuuuuuuuuucuuuccuucuuu

SEQ ID NO: 23 - RNA, 101 nt, Artificial Sequence
  misc_feature: RNA initiation site
uuuuuuuuuuuguuuuuuuuuuuuuuuuuuuuuuuuuuuuucuuuuuuuuuuuuuuuuuuuuuuuuuuuuuuccuuuuccuuuuuuuuuuauauuccuucu

SEQ ID NO: 24 - RNA, 93 nt, Artificial Sequence
  misc_feature: RNA initiation site
uuuuuuuuuuuuuugguuuuuuuuuuuuuuuuuuuuuuuuuuuuuuuuuuuuuuuuuuuuuuuccuuuuuccuuuuuuuuuuauauuccuucu

SEQ ID NO: 25 - RNA, 98 nt, Artificial Sequence
  misc_feature: RNA initiation site
uuuuuuuuuuuuuguuuuuuuuuuuuuuuuuuuuuuuuuuuuuuuuuuuuuuuuuuuuuuuuuuuuuuuuuuccuuuuuuuuuuuuuauauuccuucu

(19) United States
(12) Patent Application Publication
(10) Pub. No.: US 2001/0055756 A1
(43) Pub. Date: Dec. 27, 2001

(54) Internal de novo initiation sites of the HCV NS5B polymerase and use thereof

(76) Inventors: Charles Pellerin, Chomedey (CA); George Kukolj, Mont-Royal (CA)

Correspondence Address: STERNE, KESSLER, GOLDSTEIN & FOX P.L.L.C., Suite 600, 1100 New York Avenue, N.W., Washington, DC, 20005-3934, US

(21) Appl. No.: 09/838,386

(22) Filed: Apr. 20, 2001

Related U.S. Application Data
(60) Provisional application No. 60/198,793, filed on Apr. 21, 2000.

(51) Int. Cl. C12Q 1/70; C12Q 1/68; C07H 21/02

(52) U.S. Cl. 435/5; 536/23.72; 435/6

(57) ABSTRACT

To further define the nature of de novo initiation from the 3′-UTR, several distinct 3′-UTR's that harbor the conserved terminal 98 nucleotides, but have poly U/U-C tracts of different length were isolated and characterized. Reconstitution of de novo initiation by the mature NS5B with the different 3′-UTR RNA substrates revealed distinctively sized products that are consistent with internal initiation at specific sites within the polypyrimidine tract. These sites were mapped by demonstrating that nucleotide substitutions of the cytidylate residues in the poly U/U-C template affect the generation of specific products of the de novo initiation reaction. Moreover, initiation within the poly U/U-C template is also primed by GTP and an assay that evaluates inhibitors of this reaction as potential HCV therapeutics is claimed.

### CROSS REFERENCE TO RELATED APPLICATION

This claims priority to U.S. Provisional Application 60/198,793 filed Apr. 21, 2000, the contents of which are fully incorporated by reference herein.

### FIELD OF THE INVENTION

The present invention provides a de novo initiation site comprising a polypyrimidine tract having a cytidylate nucleotide or a poly-cytidylate (poly C) cluster located therein or adjacent thereto. This site provides a RNA template for assessing in vitro the RNA-dependent RNA polymerase (RdRp) activity of flavivirus. Particularly, the invention relates to de novo initiation sites of the NS5B protein of the hepatitis C virus and methods for identifying specific inhibitors thereof.

### BACKGROUND OF THE INVENTION

Hepatitis C virus (HCV) is the major etiological agent of post-transfusion and community-acquired non-A non-B hepatitis worldwide. It is estimated that about 170 million people worldwide are infected by the virus. A high percentage of carriers become chronically infected and many progress to chronic liver disease, so called chronic hepatitis C. This group is in turn at high risk for serious liver disease such as liver cirrhosis, hepatocellular carcinoma and terminal liver disease leading to death.

HCV is an enveloped positive strand RNA virus in the Flaviviridae family. The single strand HCV RNA genome is 9600 nucleotides in length and has a single open reading frame (ORF) encoding a single large polyprotein of about 3000 amino acids. In infected cells, this polyprotein is cleaved at multiple sites by cellular and viral proteases to produce structural and non-structural (NS) proteins. The structural proteins (C, E1, E2 and E2-p7) comprise polypeptides that constitute the virus particle (Hijikata et al., 1991; Grakoui et al., 1993(a)). The non-structural proteins (NS2, NS3, NS4A, NS4B, NS5A, NS5B) encode for enzymes or accessory factors that catalyze and regulate the replication of the HCV RNA genome. Processing of the structural proteins is catalyzed by host cell proteases (Hijikata et al., 1991). The generation of the mature non-structural proteins is catalyzed by two virally encoded proteases. The first is the NS2-3 zinc-dependent metalloprotease which auto-catalyses the release of the NS3 protein from the polyprotein. The released NS3 contains a serine protease domain at the N-terminal (Grakoui et al, 1993(b); Hijikata et al., 1993) and catalyzes the remaining cleavages from the polyprotein. The released NS4A protein has at least two roles. First, forming a stable complex with NS3 protein and assisting in the membrane localization of the NS3/NS4A complex (Kim et al., 1999) and second, acting as a cofactor for NS3 protease activity. This membrane-associated complex, in turn catalyzes the cleavage of the remaining sites on the polyprotein, thus effecting the release of NS4B, NS5A and NS5B (Bartenschlager et al., 1993; Grakoui et al., 1993(a); Hijikata et al., 1993; Love et al., 1996; reviewed in Kwong et al., 1998). The C-terminal segment of the NS3 protein also harbors nucleoside triphosphatase and RNA helicase activity (Kim et al., 1995). NS5B is an RNA-dependent RNA polymerase (RdRp) that is involved in the replication of HCV. It has been recognized that the NS3 protease and the NS5B polymerase activities constitute suitable enzymatic targets to inhibit viral replication.

The non-coding RNA regions of the HCV genome are defined by distinct 5′ and 3′ sequences (reviewed in Reed and Rice, 2000). The 5′ extremity encodes an internal ribosome-entry site that folds into a highly ordered secondary structure and directs cap-independent translation of the genomic RNA. The 3′ untranslated region is divided into three segments: (i) the highly conserved 3′-terminal 98 nucleotides (termed the X region) predicted to fold into a secondary structure of three stem-loop domains; (ii) a poly(U-U/C)-rich sequence of variable length upstream of the X-region; and (iii) further upstream is a highly variable sequence 30-40 nt in length (Kolyakhov et al., 1996; Tanaka et al., 1996).

The initial step of viral RNA replication is recognition of the 3′-end of RNA template by NS5B (RdRp), which may occur directly or indirectly with the help of cellular proteins (Lai, 1998; Strauss et al., 1999). HCV NS5B RdRp has an RNA-binding activity and preferentially binds poly(U) and poly(G) over poly(C) and poly(A) homopolymeric RNA (Yamashita et al., 1998).

It has recently been shown that NS5B can utilize the 3′-end 98-nt, X region, of the HCV genome as a minimal authentic template (Oh et al., 2000). Furthermore, this RNA was used to characterize the mechanism of RNA synthesis by the recombinant NS5B. The authors show that NS5B forms a complex with the 3′-end of HCV RNA by binding to both the poly(U-U/C)-rich and X regions of the 3′-untranslated region as well as part of the NS5B-coding sequences. Within the X region, NS5B bound stem II and the single-stranded region connecting stem-loops I and II. Furthermore, NS5B initiated RNA synthesis from a specific site within the single-stranded loop I. They conclude that HCV NS5B initiates RNA synthesis from a single-stranded region closest to the 3′-end of the X region, but do not disclose specific sequences that would induce internal de novo initiation of the NS5B polymerase.

Sun et al., 2000 and WO 2000/33635 suggest that the NS5B also catalyses de novo RNA synthesis with a HCV RNA template. They further assert that “since the viral enzyme selectively added ATP as the first nucleotide of the nascent RNA products, we conclude that HCV NS5B initiated the RNA transcription by recognizing a uridylate present in the HCV RNA fragment.” They go on to say that “it is possible that the enzyme recognized a uridylate present in the poly U/polypyrimidine tract.” However, Sun et al. fail to provide the exact sequence of the de nova RNA polymerase initiation site present in the HCV RNA genome.

In contrast to the previous reports, Applicant has found that the HCV NS5B catalyses de novo initiation of the RNA template where the resulting nascent RNA products contained GTP as the first nucleotide. In addition, Applicant has data strongly suggesting that this de novo initiation takes place internally along the RNA template, since the resulting nascent RNA products are smaller than the RNA template and correspond in length to RNA products having been initiated at distinct positions corresponding to several cytidylate clusters positioned along the poly U tract of the HCV RNA template.

These results have implications for the mechanism of HCV RNA transcription and the nature of HCV RNA templates in the infected cells. This site provides an RNA template for assessing in vitro the RdRp activity of the NS5B protein and finding specific inhibitors thereof.

De novo initiation should be closer to the authentic mechanism of RNA transcription during the disease state and in vivo infection. The exact pattern of the de novo initiation site is disclosed herein and use thereof for design of an assay to measure NS5B RdRp activity. The establishment of such an assay will facilitate the analysis of the initiation requirements and allow the testing of antiviral compounds specifically targeting de novo initiation of the HCV NS5B RNA polymerase.

### SUMMARY OF THE INVENTION

The present invention provides the initiation site for de novo (primer-independent) RNA synthesis of an RNA-dependent RNA-polymerase, particularly for a flavivirus RdRp, more particularly for the HCV NS5B polymerase.

A first embodiment of the invention provides an initiation site comprising a polypyrimidine tract having at least one cytidylate residue located therein, or adjacent thereto.

A second embodiment of the invention provides a RNA template for primer-independent RNA synthesis, this template comprising the initiation site as described in the first embodiment and a further RNA portion suitable as a template for elongation of an initiation nucleotide along said template by said polymerase.

A third embodiment of the invention provides a method of identifying a compound that inhibits primer-independent de novo RNA synthesis catalyzed by the HCV NS5B polymerase.

A fourth embodiment of this invention provides a method of inhibiting primer-independent de novo RNA synthesis catalyzed by HCV NS5B polymerase where the compound is identified by the method as described in the third embodiment of this invention.

A fifth embodiment of this invention provides a method of inhibiting the replication of hepatitis C virus where the compound is identified by the method as described in the third embodiment of this invention.

A sixth embodiment of this invention provides a method for producing an anti-HCV compound whereby the compound is identified according to the method as described in the third embodiment of this invention.

### BRIEF DESCRIPTION OF THE DRAWINGS/FIGURES

FIG. 1 is a schematic representation of the HCV 3′ ends used as RNA templates for the NS5B polymerase. HCV 3′ end cDNAs were cloned downstream of a T7 RNA polymerase promoter and, following XbaI digestion and Mung Bean Nuclease treatment of DNA, RNA was synthesized in vitro as described in Materials and Methods. For each RNA, the number at the right indicates the total length of the RNA while the numbers under the sequences indicate the nucleotide length of the different sections of the RNA. From 5′ to 3′ the RNAs contain a short leader of vector sequence followed by the 3′-end of the NS5B coding region, the NS5B stop codon and the HCV 3′ variable sequence; the polypyrimidine tract (that varies among the different RNA templates) and the highly conserved 3′-X region terminates the RNA templates.

FIG. 2A shows the major RNA products generated by HCV NS5B and the 3′-UTR RNA produced that are shorter than the input template. Products generated by NS5B polymerase with RNA templates: 24 (lane 2), 24-1 (lane 3), 128-4 (lane 4), 130-21 (lane 5) or 150-41 (lane 6) were resolved a denaturing 8 M urea/5% polyacrylamide gel. RNA molecular weight standards (M, lane 1) were used to interpolate the size of products. The filled circles represent the predicted location of radioactive products that correspond to full length RNA complementary to the template strand (i.e. de novo initiated at the 3′-end and run off at the 5′ end of the template strand). The arrows highlight the major radioactive products detected.

FIG. 2B is a schematic representation of the distance from the C stretches (2 Cs or more) present in the poly U/UC and the 5′ end of templates 24, 24-1 and 128-4; the distances correspond to the length of the major products from each of these templates and represent sites of internal de novo initiation by the HCV NS5B.

FIG. 3 is a schematic representation of the 3 mutated versions of template 24-1 by site-directed substitution of the “CCCC” and/or the “CC” sequences from template 24-1 and the predicted effect on generation of HCV NS5B polymerization products. Non-substituted template 24-1 in a standard NS5B reaction produces 227 and 246 nt products. Template 24-1(m1) has the 5′-proximal “CCCC” sequence mutated to “UUUU”. In template 24-1(m2), the “CC” within the poly U/C is mutated to “UU”. Template 24-1(dm) encodes the double mutant with both m1 and m2 modifications.

FIG. 4 shows a 8 M urea/5% acrylamide gel electrophoresis analysis of the reaction products using the mutants RNA templates as represented in FIG. 3. Nucleotide numbers shown on the right correspond to the two major products (246 and 227 nt) as described to originate from the 24-1 RNA template. The size of the RNA markers (lane M) is indicated on the left.

FIG. 5 shows the RNA products generated by NS5B polymerase in the presence of [γ³²P]GTP or [γ³²p]ATP. De novo initiation reactions containing 25 nM (panels A, B and D) or 100 nM (panels C and E) of NS5B polymerase and 50 nM of RNA templates 24-1 (lanes 1), 24-1 (dm) (lanes 2) or 128-4 (lanes 3) were used in polymerase reactions in the presence of either [α³³P]UTP (panel A), [γ³²P]GTP (panels B and C) or [γ³²P]ATP (panels D and E). The products were analysed on a denaturing 8 M urea/5% acrylamide gel. Lane (M) displays radioactively labeled RNA molecular weight markers

FIG. 6 demonstrates the effect of GTP analog stimulation of NS5B polymerase in the de novo initiation reaction with the 24-1 HCV RNA template. Various GTP analogs were added, at a final concentration of 500 μM, to the NS5B polymerization reactions that contained: 100 μM (hatched bars) or 500 μM GTP (solid bars). Measurement of the stimulation by the analogs was normalized to the activity obtained with GTP alone. Reactions containing 600 μM or 1 mM GTP were also performed [labeled as the +500 μM GTP series]. Reactions were incubated for 2.5 h in the presence of 5 nM NS5B and 10 nM of RNA template 24-1. The effect of the various analogs on the reaction is represented by the fold-stimulation in activity relative to the activity obtained with 100 μM or 500 μM GTP, respectively (these two are reference reaction bars). Note that guanosine, GpppG and 7-methyl GpppG all stimulate the de novo initiation reaction. GTP 2′3′dialdehyde and ribavirin triphosphate are inhibitory. 5′-p-FSBA =5′-p-Fluorosulfonyl-benzoyladenosine.

FIG. 7 demonstrates the utility of one of the claimed RNA templates (24-1) in a HCV polymerase de novo initiation reaction that measures the dose-dependent inhibition of compound I. Panel A: table summarizing the quantification of NS5B synthesized RNA from the 24-1 template in the presence of indicated amounts of compound I, as determined through a DE-81 filter capture assay described in the materials and methods. Panel B: plot of the %-inhibition versus compound concentration and the determination of the IC₅₀ value through non-linear regression analysis using the Hill equation:100−[(countᵢₙₕ−count_{blank})/(count_{ctl}−count_{blank})×100]. Panel C: RNA products resolved and visualized following electrophoresis in 8 M urea/5% acrylamide gels.

### DETAILED DESCRIPTION OF THE INVENTION

The following detailed description of the invention is provided to aid those skilled in the art practicing the present invention. Even so, the following detailed description should not be construed to unduly limit the present invention as modifications and variations in the embodiments discussed herein can be made by those of ordinary skill in the art without departing from the spirit or scope of the present inventive discovery.

The contents of each of the references cited herein are herein incorporated by reference in their entirety.

### Definitions

Amino acid residues described herein are preferred to be in the “L”=0 isomeric form. However, residues in the “D” isomeric form may be substituted for any L-amino acid residue, provided the desired properties of the polypeptide are retained.

All amino-acid residue sequences represented herein conform to the conventional left-to-right amino-terminus to carboxy-terminus orientation.

All nucleotide sequence represented herein conform to the conventional left-to-right 5′ end to 3′ end orientation.

Conventional methods of gene isolation, molecular cloning, vector construction, etc., are well known in the art and summarized, for example, in Sambrook, J. et al., Molecular Cloning: A laboratory Manual, Second Edition, Cold Spring Harbor Laboratory Press, Cold Spring Harbor, N.Y., and Ausubel et al., Current Protocols in Molecular Biology, John Wiley & Sons, Inc. One skilled in the art can readily reproduce the plasmid vectors described herein without undue experimentation employing these methods. The various nucleic acid sequences, fragments, etc., necessary for this and other purposes can be readily obtained as components of commercially available plasmids, or are otherwise well known in the art and publicly available, or readily reproducible based upon published information.

The phrase “consisting essentially of” when referring to a particular nucleotide or amino acid means a sequence having the properties of a given SEQ ID No. For example, when used in reference to a particular sequence, the phrase includes the sequence per se and molecular modifications that would not affect the basic and novel characteristics of the sequence.

The term “cluster” as used herein defines a stretch of two or more adjacent similar nucleotides aligned consecutively.

The terms “de novo” or “primer-independent” RNA synthesis are used interchangeably and refer to the ability of a polymerase to bind to a specific template, to prime RNA synthesis using a first initiating nucleotide triphosphate (NTP) complementary to the initiation site, and elongate/extend the first nucleotide to transcribe the template without the help of an extraneous oligonucleotide primer complementary to the template.

A “derivative” of the HCV NS5B polypeptide or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, and may or may not alter the essential activity of the original HCV NS5B polypeptide. As mentioned above, the HCV NS5B polypeptide or protein of the invention includes any analogue, fragment, derivatives or mutant which is derived from a HCV NS5B polypeptide and which retains at least one property or other characteristic of the HCV NS5B polypeptide.

The terms “elongation” or “extension” are used interchangeably and mean the consecutive addition of nucleotides as directed by a complementary template of DNA or RNA that is carried out by an appropriate polymerase. In the particular context of this invention, elongation or extension is carried out on an RNA template by a flavivirus RNA-dependent RNA polymerase, particularly the HCV NS5B RdRp.

An “expression operon” refers to a nucleic acid segment that may possess transcriptional and translational control sequences, such as promoters, enhancers, translational start signals (e.g., ATC or AUG codons), polyadenylation signals, terminators, and the like, and which facilitate the expression of a polypeptide coding sequence in a host cell or organism.

The term “functional” as used herein implies that the nucleic or amino acid sequence is functional for the recited assay or purpose.

A “fragment” or “portion” of the HCV NS5B polypeptide means a stretch of amino acid residues of sufficient length or an NS5B polypeptide having amino acids deleted therein, while retaining at least one of its function such as binding to a template, priming, or elongation along a template.

The term “initiation site” means a site where the polymerase recognizes a cytidine nucleotide or a nucleotide sequence comprising at least one cytidine moiety. The initiation cytidylate nucleotide functions as a recognition site for primer-independent de novo RNA synthesis on an RNA template catalyzed by HCV RNA-dependent RNA polymerase.

The term “initiation” refers the first step of RNA synthesis, that incorporates the initial 5′ position nucleotide of the nascent RNA chain. This reaction is also referred to as “priming”.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production. For example, an “isolated nucleic acid” may comprise a DNA molecule inserted into a vector, such as a plasmid or virus vector, or integrated into the genomic DNA of a prokaryotic or eukaryotic cell or host organism.

The terms “isolated protein” or “isolated and purified protein” refer primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

“Nucleic acid” or a “nucleic acid molecule” as herein refers to any DNA or RNA molecule, either single or double stranded and, if single stranded, the molecule of its complementary sequence in either linear or circular form.

The term “NS5B” refers to a portion of the HCV genome located near the 3′ end of the viral genome that specifies the region encoding a protein, termed the “NS5B protein”, “NS5B polypeptide”, “NS5B polymerase” or combinations of these terms which are used interchangeably herein. NS5B in its natural state, functions as an RNA-dependent RNA polymerase (RdRp). The nucleic acid region encoding the NS5B protein may also be referred to as the “NS5B gene”. Thus, the term “NS5B” may refer to either a nucleic acid molecule encoding the NS5B polypeptide, to an NS5B gene or to an NS5B polypeptide, or to any portions thereof, depending on the context in which the term is used. NS5B may further refer to natural allelic variants, mutants and derivatives of either NS5B nucleic acid sequences or NS5B polypeptides. The NS5B nucleic acid, NS5B gene or NS5B protein referred to is a functional polymerase, or to a non-functional polymerase that still binds to an appropriate template.

The term “oligonucleotide”, as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “percent similarity”, “percent identity” and “percent homology” when referring to a particular sequence are used as set forth in the University of Wisconsin GCG software program.

As used herein, the terms “polypyrimidine tract” and “poly U” are used interchangeably and refer to a stretch of consecutive pyrimidine nucleotides essentially consisting of uridylate residues. Preferably, the poly U stretch essentially consists of ≧70% uridylate residues. More preferably, the poly U tract essentially consists of ≧80% uridylate residues. Most preferably, the poly U tract essentially consists of ≧90% uridylate residues.

As used herein the terms “poly U/C” and “poly U/U-C tract” are used interchangeably and refer to a poly U tract as defined above optionally interrupted with at least one cytidylate residue, preferably two or more cytidylate residues and more preferably one or more cluster of cytidylate residues.

The term “plasmid” refers to an extrachromosomal genetic element. The starting plasmids herein are either commercially available, publicly available on an unrestricted basis, or can be constructed from available plasmids in accordance with published procedures. In addition, equivalent plasmids to those described are known in the art and will be apparent to the ordinarily skilled artisan.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be elongated (extended) at its 3′ terminus by the addition of nucleotides by the action of a polymerase or similar activity to yield a primer elongation (extension) product. The primer may vary in length depending on the particular conditions and requirement of the application. For example, in diagnostic applications, the oligonucleotide primer is typically 15-25 or more nucleotides in length. The primer must be of sufficient complementary to the desired template to prime the synthesis of the desired extension product, that is, to be able anneal with the desired template strand in a manner sufficient to provide the 3′ hydroxyl moiety of the primer in appropriate juxtaposition for similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequences has sufficient complementarity with the sequence of the desired template strand to functionally provide a template-primer complex for the synthesis of the extension product.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method.

“Recombinant DNA cloning vector” as used herein refers to any autonomously replicating agent, including, but not limited to, plasmids and phages, comprising a DNA molecule to which one or more additional DNA segments can or have been added.

The terms “RNA synthesis” and “transcription” are used interchangeably and are defined by the specific steps taken by an RNA polymerase of: recognizing and binding to a template initiation site; priming by incorporating a first complementary nucleotide; and adding consecutively complementary nucleotides to elongate the nascent RNA chain.

A “replicon” is any genetic element, for example, a plasmid, cosmid, bacmid, phage or virus, that is capable of replication largely under its own control. A replicon may be either RNA or DNA and may be single or double stranded.

The term “specifically hybridize” refers to the association between two single-stranded nucleic acid molecules of sufficiently complementary sequence to permit such hybridization under-pre-determined conditions generally used in the art (sometimes termed “substantially complementary”). In particular, the term refers to hybridization of an oligonucleotide with a substantially complementary sequence contained within a single-stranded DNA or RNA molecule of the invention. To the substantial exclusion of hybridization of the oligonucleotide with single-stranded nucleic acids of non-complementary sequence.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, to that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin biding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

The terms “transform”, transfect”, “transduce”, shall refer to any method or means by which a nucleic acid is introduced into a cell or host organism and may be used interchangeably to convey the same meaning. Such methods include, but are not limited to, transfection, electroporation, micro-injection, PEG-fusion and the like.

The term “template” refers to an oligonucleotide of DNA, or preferably RNA, that serves as one of the substrate for a polymerase. The sequence of a template is complementary to the sequence produced by the polymerase during transcription.

Different “variants” of the HCV NS5B polypeptide exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acids residues are substituted with conservative or non-conservative amino acids, (b) variants in which one or more amino acids are added to the HCV NS5B polypeptide, (c) variants in which one or more amino acids include a substituent group, and (d) variants in which the HCV NS5B polypeptide is fused with another peptide or polypeptide such as a fusion partner, a protein tag or other chemical moiety, that may confer useful properties to the HCV NS5B polypeptide, such as, for example, an epitope for an antibody, a polyhistidine sequence, a biotin moiety and the like. Other HCV NS5B polypeptides of the invention include variants in which amino acid residues from one species are substituted for the corresponding residue in another species, either at the conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to the person having ordinary skill in the art. To the extent such allelic variations, analogues, fragments, derivatives, mutants, and modifications, including alternative nucleic. acid processing forms and alternative post-translational modification forms result in derivatives of the HCV NS5B polypeptide that retain any of the biological properties of the HCV NS5B polypeptide, they are included within the scope of this invention.

The term “vector” as used herein refers to a nucleic acid compound used for introducing exogenous DNA into host cells. A vector comprises a nucleotide sequence which can encode one or more protein molecules. Plasmids, cosmids, viruses, and bacteriophages, in the natural state or which have undergone recombinant engineering, are examples of commonly used vectors, to which another genetic sequence or element (either DNA or RNA) may be attached so as to bring about the replication of the attached sequence or element.

### Preferred Embodiments

According to the first embodiment of this invention, the initiation site for de novo (primer-independent) RNA synthesis of the HCV NS5B RdRp preferably comprises a polypyrimidine tract having two or more adjacent cytidylate residues located therein, or adjacent thereto.

Preferably, the RNA initiation site comprises a polypyrimidine tract having a cluster of cytidylate residues located therein, or adjacent thereto.

Preferably, the RNA initiation site comprises a polypyrimidine tract having one or more cluster of cytidylate residues located therein, or adjacent thereto.

More preferably, the RNA initiation site comprise a sequence selected from the group consisting of: (P)ₙ(C)ₘ; (C)ₘ(P)ₙ; or (P)ₙ(C)ₘ(P)ₙ, wherein P is a pyrimidine or an analog thereof, each n is independently 2 to 200 and m is 1 to 10.

Alternatively, the RNA initiation site preferably comprises a CCC or CCCC sequence adjacent to a polypyrimidine tract.

Still, most preferably, the RNA initiation site comprises a sequence selected from the group consisting of:

U₁₃CU₃CCUUCU₃ (SEQ ID NO. 22);

U₁₁GU₂₉CU₃₀CCU₄CCU₁₀AUAUUCCUUCU (SEQ ID NO. 23);

U₁₄GGU₄₇CCU₅CCU₁₀AUAUUCCUUCU (SEQ ID NO. 24); and

U₁₃GU₅₈CCU₁₃AUAUUCCUUCU (SEQ ID NO. 25).

Preferably, the polypyrimidine tract is a poly(U) tract consisting of equal to, or greater than 70% of uridylate residues, more preferably 80%, most preferably 90%.

According to the second embodiment of the invention provides a RNA template for primer-independent RNA synthesis, this template comprising the initiation site as described in the first embodiment and a further RNA portion suitable as a template for said polymerase elongation.

Preferably, the RNA template comprises a polypyrimidine tract having at least one cytidylate residue located therein, or adjacent thereto. More preferably, the initiation site of this template comprises two or more cytidylate residues, most preferably one or more cluster of cytidylate residues.

Alternatively, the RNA template comprises a sequence of one or more cytidylate residue adjacent to, the polypyrimidine tract, preferably two or more C residues, more preferably a CCC sequence and most preferably a CCCC sequence upstream of the polypyrimidine tract.

According to the third embodiment of the invention, there is provided a method of identifying a compound that inhibits primer-independent de novo RNA synthesis catalyzed by the HCV NS5B polymerase, comprising the steps of:

a) contacting a RNA template as described above, with the NS5B polymerase in the absence of a primer and in the absence of the compound under conditions permitting RNA synthesis, and determining the amount of RNA thus formed;

b) contacting a RNA template as in a), with said NS5B polymerase in the absence of a primer and in the presence of the compound under the same conditions as in a), and determining the amount of RNA thus formed; and

c) comparing the amount of RNA product formed in b) with that of a);

wherein any reduction in the amount of RNA product formed in b) compared with that formed in a) indicates a compound that is an inhibitor of primer-independent de novo RNA synthesis catalyzed by the HCV NS5B polymerase.

Preferably, the compound inhibits binding of the NS5B polymerase to the initiation site.

Alternatively, the compound inhibits priming of the NS5B polymerase once bound to the initiation site.

As a further alternative, the compound inhibits elongation by the RNA polymerase along the template.

According to the fourth embodiment of this invention, there is provided a method of inhibiting primer-independent de novo RNA synthesis catalyzed by HCV NS5B polymerase comprising the step of:

contacting the polymerase with a polymerase-inhibiting effective amount of a compound that inhibits primer-independent de novo RNA synthesis catalyzed by the polymerase,

wherein the compound is identified by the method as described in the third embodiment of this invention.

Preferably, the method provides inhibition of the binding of the NS5B polymerase to the initiation site.

Alternatively, the method provides inhibition of priming of the NS5B polymerase once bound to the initiation site.

As a further alternative, the method provides inhibition of elongation by the RNA polymerase along the template.

According to the fifth embodiment of this invention provides a method of inhibiting the replication of hepatitis C virus comprising the step of:

contacting the hepatitis C virus with an antiviral effective amount of a compound that inhibits primer-independent de novo RNA synthesis catalyzed by HCV NS5B polymerase,

where the compound is identified by the method as described in the third embodiment of this invention.

Preferably, the method provides inhibition of the binding of the NS5B polymerase to the initiation site.

Alternatively, the method provides inhibition of priming of the NS5B polymerase once bound to the initiation site.

As a further alternative, the method provides inhibition of elongation by the RNA polymerase along the template.

A sixth embodiment of this invention provides a method for producing a anti-HCV compound comprising the step of:

identifying the compound according to the method as described in the third embodiment of this invention.

### EXAMPLES

### Materials and Methods

### Cloning of the HCV 3′ Sequences

The template 24 cDNA sequence (SEQ ID NO. 1), complementary to the HCV plus strand 3′-end, was obtained by semi-nested RT-PCR performed on RNA extracted from the serum of an infected individual (HCV genotype 1b). The sequence of the 3 oligonucleotides used in the RT-PCR were:

5087: 5′-TCT AGA CAT GAT CTG CAG AGA GGC CAG TAT CAG CAC TCT C-3′ (antisense), (SEQ ID NO.2), 6089: 5′-ATG GGG CAA AGG ACG TCC G-3′ (external sense) (SEQ ID NO.3) and 8017: 5′-GGA CCA AGC TTA AAC TCA CTC CAA TCC-3′ (internal sense) (SEQ ID NO.4).

The final PCR product was then directly cloned into the pCR3 vector (Invitrogen) downstream of the T7 RNA polymerase promoter. From this cloned cDNA, a HindIII fragment was removed (essentially consisting of vector sequences present between the T7 promoter and the HCV sequences). The resulting DNA was used to synthesize template 24-1 (SEQ ID NO.5). The cDNA sequence encoding templates 128-4 (SEQ ID NO.6), 130-21 (SEQ ID NO.7) and 150-41 (SEQ ID NO.8) were generated by combining the 5′-portion (upstream of the poly U/U-C tract) of template 24-1 cDNA with three different cDNA fragments obtained by semi-nested RT-PCR performed on RNA extracted from an HCV infected liver (unknown genotype). The oligonucleotides used for the amplifications were 5087 (SEQ ID NO. 2), 8046: 5′-TCC ACA GTT ACT CTC CAG-3′ (external sense) (SEQ ID NO. 9) and 8038: 5′-TAG GCA TTT ACC TGC TCC CCA ACC-3′ (internal sense) (SEQ ID NO.10).

### In Vitro Synthesis Of The RNA Templates

Plasmid DNA containing the 3′ HCV cDNA was linearized with XbaI and then treated with Mung bean nuclease to eliminate extraneous overhanging DNA such that the run-off transcript synthesized by T7 RNA polymerase terminated with the authentic HCV 3′-end. RNA synthesis was performed, using the T7 RiboMAX large scale RNA production system (Promega), for 2-2.5 h at 37° C. followed by DNase digestion for 30 min at 37° C. After phenol-chloroform extraction and isopropanol precipitation, the pelleted RNA was resuspended in DEPC-treated water and passed through a Microspin G-50 column (Pharmacia). RNA concentration was determined by OD260/280 measurement. Some of the RNA templates were size fractionated and purified by capillary gel electrophoresis (Beckman Coulter P/ACE MDQ) on gels of 4 % (w/v) hydroxyethylcellulose, 20 mM TAPS, 7 M Urea, pH 6.3. Homogeneous full-length RNA templates were used as substrates to confirm the generation of internal sites of de novo initiation by the NS5B polymerase.

### Purification of Mature HCV NS5B Polymerase

The NS5B polymerase was produced as a hexa-histidine tagged precursor in Sf-21 insect cells infected from a recombinant baculovirus construct (BacHTaA5B). This vector encodes the full-length HCV NS5B and an N-terminal hexa-histidine linked by a dodecapeptide motif that constitutes an NS5A/NS5B cleavage site. Processing of the precursor protein with the heterodimeric NS3 protease/NS4A peptide-cofactor (Bartenschlager 1999) produces a mature form of the 591 amino acid NS5B (SEQ ID NO. 12). In summary, BacHTaA5B infected Sf-21 cell pellets were resuspended in lysis buffer (25 mM Tris pH 7.5, 1 mM EDTA, 5 mM MgCl₂, 2 mM β-mercaptoethanol, 500 mM NaCl, 50% glycerol, 2% n-dodecyl-β-D-maltoside and a cocktail of protease inhibitors), Dounce homogenized, treated with DNase I, sonicated and then clarified by centrifugation (105000 ×g, 45 min., 4° C.). The resulting supernatant was diluted with 3 volumes of buffer A (25 mM Tris pH 7.5, 2 mM β-mercaptoethanol, 10% glycerol,10 mM imidazole, 500 mM NaCl, 0.15% dodecyl-β-D-maltoside and a cocktail of protease inhibitors) and applied to a Ni-NTA chelating resin (Qiagen). The HTaA5B protein was eluted by a linear (10-500 mM) imidazole gradient in buffer A, and then diluted with buffer B (20 mM Tris pH 7.5, 20% glycerol, 2 mM β-mercaptoethanol, 1 mM EDTA, 0.15% n-dodecyl-β-D-maltoside) to reduce the NaCl concentration to 300 mM. The HTaA5B was applied to a DEAE Sepharose column, to remove nucleic acids and the flow-through was diluted two-fold with buffer B to further reduce the NaCl concentration to 150 mM for the subsequent Hi-trap heparin chromatography. Purified HTaA5B was eluted with a 200-1000 mM NaCl gradient from the Hi-trap heparin column.

The NS3/4A cleavage (Laplante et al., 2000) that generates the mature NS5B uses a 1:50:1.25 molar ratio of NS3 protease: 4A cofactor peptide: HTaA5B precursor in buffer B diluted with an equal volume of buffer C (50 mM NaPO₄ pH 7.8, 10% glycerol, 0.3 M NaCl, 0.1% n-dodecyl-β-D-maltoside). The reaction is performed at room temperature for 45 min. followed by a 5 hour incubation at 4° C. Following NS3/NS4A catalyzed removal of the His-tag, the reaction mixture is supplemented with 10 mM imidazole and batch-mixed with Ni-NTA resin to bind the cleaved His-tag tails and any uncleaved HTaA5B protein. The resin is pelleted by centrifugation and the supernatant (mature NS5B fraction) is subjected to Hi-trap heparin chromatography as described above to separate the NS3 protease from NS5B RdRp. The NS5B fractionated by heparin chromatography is applied, in buffer B containing 800 mM NaCl, to a preparative Superose-12 gel filtration column to recover a highly pure NS5B.

### Site-Directed Mutagenesis

Mutations within the poly (U/UC) were generated with the QuickChange Site-Directed Mutagenesis Kit (Stratagene) using the 24-1 cDNA clone as template for PCR. The following complementary pairs of oligonucleotides were used to generate:

```
          (i) Mutant 24-1(ml): 5′-CCA ATA GGC CAT TTT TTT TTT TTT
TTT TTC TTT CCT TCT TTG GTG-3′ (SEQ ID NO. 13) and 5′-GGA AAG
AAA AAA AAA AAA AAA AAA TGG CCT ATT GGC CTG GAG-3′ (SEQ ID NO. 14);
          (ii) Mutant 24-1(m2): 5′-CCC CTT TTT TTT TTT TTC TTT TTT
TCT TTG GTG GCT CCA TC-3′ (SEQ ID NO. 15) and 5′-GCC ACC AAA
GAA AAA AAG AAA AAA AAA AAA AGG GGA TGG CC-3′ (SEQ ID NO. 16); and
          (iii) Mutant 24-1(dm): 5′- CCA ATA GGC CAT TTT TTT TTT TTT
TTT TTC TTT TTT TCT TTG GTG-3′ (SEQ ID NO. 17) and 5′-CAC CAA
AGA AAA AAA GAA AAA AAA AAA AAA AAA ATG GCC TAT TGG-3′ (SEQ ID NO. 18).
```

### HCV NS5B RNA Polymerization Reactions

Unless indicated otherwise, the standard reactions that incorporated [α-³³P]UTP contained 5 nM of purified NS5B and 10-50 nM of the indicated RNA template (i.e.: 24, 24-1, 128-4, 130-21, 150-41 or mutant templates) incubated at 22° C. for 2.5 h in 22.5 mM Tris-HCl pH 7.5, 5 mM MgCl₂, 1 mM EDTA, 1.5 mM DTT, 30 mM NaCl, 0.33% N-Dodecyl-β-D-Maltoside, 0.01% Igepal CA-630, 5% DMSO, 5 μCi [α-³³P]UTP, 10 μM UTP, 500 μM of the three other rNTPs and 0.16 U/μl RNase inhibitor (Promega). NS5B polymerase reactions that incorporated γ-³²P into the radioactively labeled 5′product were supplemented with either 8 μCi of [γ-³²P]GTP or [γ-³²P]ATP in place of [α-³³P]UTP in the standard reaction. The reactions were then supplemented with PK buffer (50 mM Tris HCl pH 7.5 150 mM NaCl, 0.5% SDS) and treated with Proteinase K (0.8 μg /μl) and glycogen (0.1 μg/μl) for 30 min at 37° C. RNA was extracted with phenol-chloroform and precipitated with isopropanol. The RNA pellet was dissolved in 10-15 μl of denaturing solution (80% formamide, 10 mM EDTA), heated for 10 min at 70-75° C. and analysed on denaturing 8 M urea/5% polyacrylamide gels. Gels were dried and radioactive products visualized with a phospho-imager (Molecular Dynamics).

### Testing of GTP Analogs And Other Inhibitors In The De Novo Initiation Reaction

m7GpppG and GpppG were purchased from Ambion; Ribavirin monophosphate and Ribavirin triphosphate were purchased from Chemgenes (Ashland, Mass.); ddGTP was from Amersham; Caged GTP was from Molecular Probes. All other GTP analogs were from Sigma/Aldrich. Each of the analogs was added (at 500 μM) to the NS5B polymerization reaction containing either: 100 μM or 500 μM GTP (reference reactions). Stimulation by GTP itself, as a positive control, was performed such that the reactions then contained 600 μM or 1 mM GTP (respectively labeled as the +500 μM GTP bars). The standard polymerase reactions incorporating [α-³³P]UTP were incubated for 2.5 h in the presence of 5 nM NS5B and 10 nM of RNA template 24-1. The reactions were stopped by spotting 8 μl on a DE81 filters which were then washed extensively (3×) with 1M NaPO₄ pH 7 and dried. The radioactivity incorporated into product RNA that was retained on the DE-81 filters was measured in a scintillation counter. The effect of the various analogs on the reaction is represented by the fold stimulation of activity compared to 100μM or 500μM GTP (reference reaction bars). 5′-p-FSBA=5′-p-fluorosulfonyl-benzoyladenosine

### Example 1

### Template Motifs in the Poly U/C Segment Correspond to Sites of De Novo Initiation

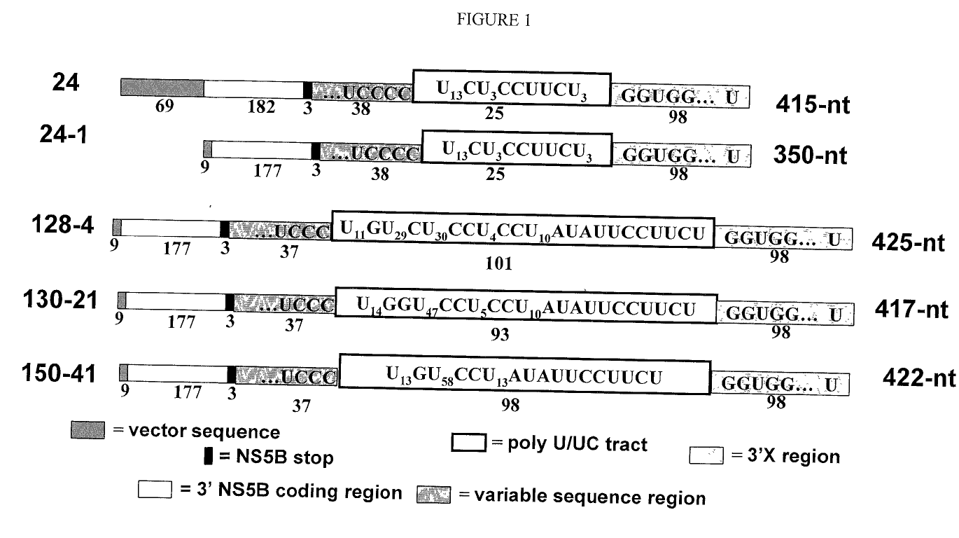

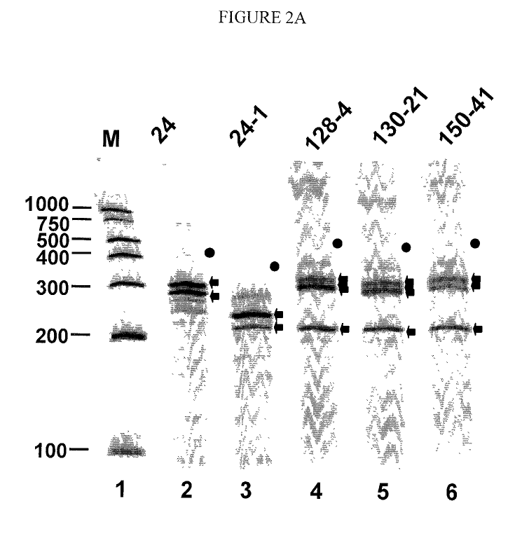

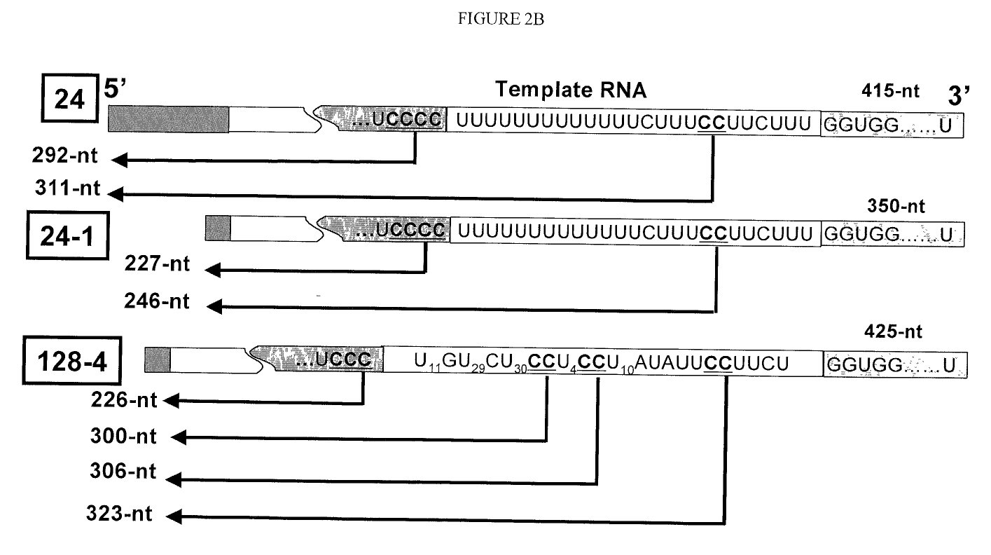

Primer-independent, de novo initiation of RNA synthesis by the HCV NS5B polymerase occurs on templates of various origin (Oh et al., 1999; Oh et al., 2000; Zhong et al., 2000; Luo et al., 2000; and WO 2000/33635). We have confirmed this observation both with templates that mimic the 3′-end of the plus-strand genome and the 3′-end of the minus-strand anti-genome. In contrast to the products that are generated by NS5B with non-specific RNA templates (Luo et al., 2000) or RNA templates that mimic the 3′-end of the minus-strand, the predominant products from templates that correspond to the 3′-end of the plus-strand are significantly shorter than the unit length template. FIG. 1 schematically displays the various RNA substrates with highlighted regions of the 3′-UTR, that were used in this study. The only distinction between templates 24 and 24-1 is the length of RNA upstream of the poly-pyrimidine (poly U/U-C) tract. The remainder of the RNA templates (128-4, 130-21 and 150-41) are slight variations of the 24-1 sequence that contain poly U/U-C tracts of various length that were amplified and cloned from an HCV infected liver. HCV NS5B polymerase reactions that used the 415 nt RNA template 24, consistently produced two major radioactive products of approximately 292 and 311 nt (FIG. 2A, lane 2 denoted with arrows). A 415 nt product that corresponds to a run-off transcript initiating at the terminal nucleotide of the 3′-template was produced (FIG. 2A, lane 2 denoted by filled circle), but to a significantly lesser extent.

The origin of the RNA products that are shorter than the input template was investigated through the use of slightly modified RNA templates. Interestingly, the sub-template size products were specific to reactions that contained templates mimicking the 3′-end of the HCV plus-strand. In reactions that used the 3′-end of the HCV minus-strand as a template (341 nts), none of the major products were shorter than the input template (data not shown; Oh et al., 1999). The possible origin of non-degraded products that were shorter than input template may be from: (i) premature termination of RNA polymerization that initiated de novo at the 3′ terminal nucleotide of the template; or (ii) run-off transcription that initiated at sites remote (upstream) from the 3′-end of the template. In order to distinguish between these two possibilities, we slightly modified template 24. An alteration at the 5′-end constituted template 24-1 and reduced the length of RNA upstream of the poly U/U-C tract from 292 to 227 nucleotides, −a 65 nt difference. The two major products generated by HCV polymerase with the truncated 24-1 template were approximately 227 and 246 nt long (FIG. 2A, lane 3 highlighted by two arrows). These products were also shorter than predicted from the complete transcription of a 350 nt unit length template (FIG. 2A, lane 3 filled circle), and consistent with the results obtained with template 24. Moreover, the difference in length between the pair of products generated from template 24 and 24-1 was approximately 65 nt. This differential coincides with the length of the alteration that distinguishes the 5′-end of template 24 from template 24-1 and suggests that the pair of major products resulted from HCV polymerase directed transcription that initiated at internal sites and ran-off the different 5′-ends. We were careful to ensure that the template preparation contained full length RNA template (and was not contaminated with a small amount of RNA that may have prematurely terminated during substrate preparation by the T7 RNA transcription) by employing high resolution capillary gel electrophoresis to purify full length template RNA substrate.

### Example 2

### Position Of The Poly U/C Initiation Sites From The 5′-End Predicts The Length Of The Synthesized Run-Off RNA Product

In a further effort to localize the potential initiation sites within the HCV 3′ plus-strand UTR, we modified the 24-1 RNA to generate templates 128-4, 130-21, and 150-41. The three templates and 24-1 share the similar sequence upstream and downstream of the poly-pyrimidine tract, but have the poly U/U-C portion increased from 25 to 101, 93, and 98 nucleotides, respectively (FIG. 1). In polymerization reactions using the mature HCV NS5B, the major products generated from each of these templates were shorter than input template RNA (FIG. 2A, lanes 4-6, compare position of arrows with location of filled circles). At 425-nt, template 128-4 was the longest model substrate used in these studies. Four major products (with estimated nucleotide lengths: 226, a 300/306 doublet, and 323) were generated by HCV NS5B from the 128-4 RNA. Assuming a run-off transcript that terminates with a sequence complementary to the 5′ end of the template, the 226, 300, 306, and 323 products are all predicted to initiate at template cytidylate doublets within the 128-4 template polypyrimidine tract (FIG. 2B). The 226 nt product initiates at the stretch of cytidylates 5′ to the poly U/U-C tract, and migrates slightly quicker than the 227 nt product generated by template 24-1 (which encodes a 4 cytidylate stretch rather than the 3 C stretch encoded by the three other templates). The intense band migrating in the range of 300-310 nt range corresponds to a predicted doublet originating from the two pairs of cytidylates in the middle of the poly U/C tract; and the 323 nt product represents RNA initiated at the 3′ CC motif in the poly U/C tract.

### Example 3

### mutational analysis

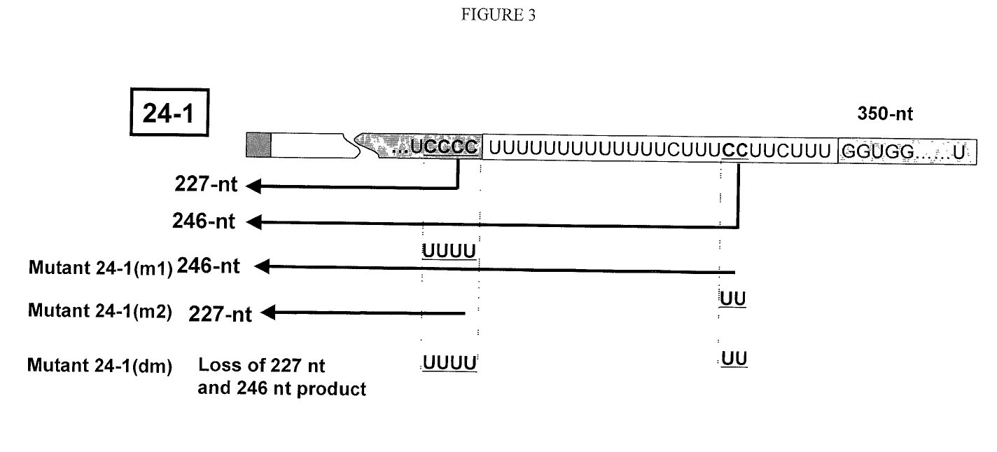

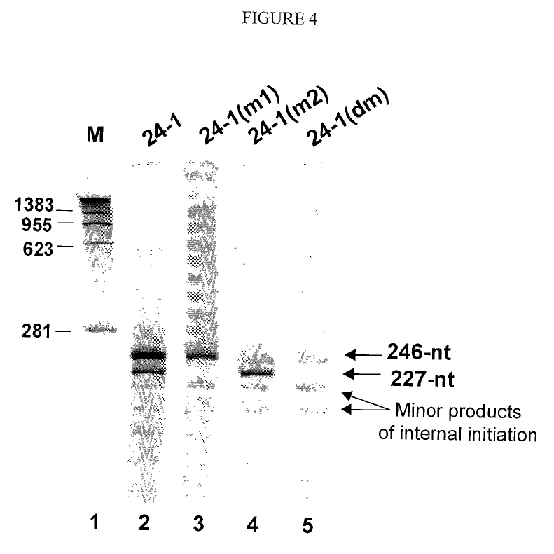

In order to establish a role for the template ‘CC’ motifs as potential sites of initiation on the HCV 3′ UTR, the two CC motifs of template 24-1 were replaced with U residues. FIG. 3 depicts the three mutant templates used in this example. Template 24-1 (m1) (SEQ ID NO. 18) harbors a four nucleotide substitution at the 5′ portion of the poly U/C tract that was predicted to abolish initiation and generation of the 227 nt product. Template 24-1 (m2) (SEQ ID NO. 19) has a two nucleotide substitution of the CC motif in the poly U/C tract that was predicted to eliminate the 246 nt product, and RNA template 24-1(dm) (SEQ ID NO. 20) harbors both the (m1) and (m2) mutations. FIG. 4 displays the products of primer-independent RNA synthesis by the HCV NS5B with each of these templates. Relative to unmodified 24-1, the 24-1(m1) template does not generate the 227 nt product. The 24-1(m2) template substantially reduced the production of 246 nt RNA, and the 24-1 (dm) double mutant significantly decreased the production of both 246 and 227 nt species. Minor, less intense products of internal initiation are still visible and represent products that were initiated at alternative cytidylates within the template.

### Example 4

### A Role For GTP in Primer-Independent RNA Synthesis By HCV NS5B

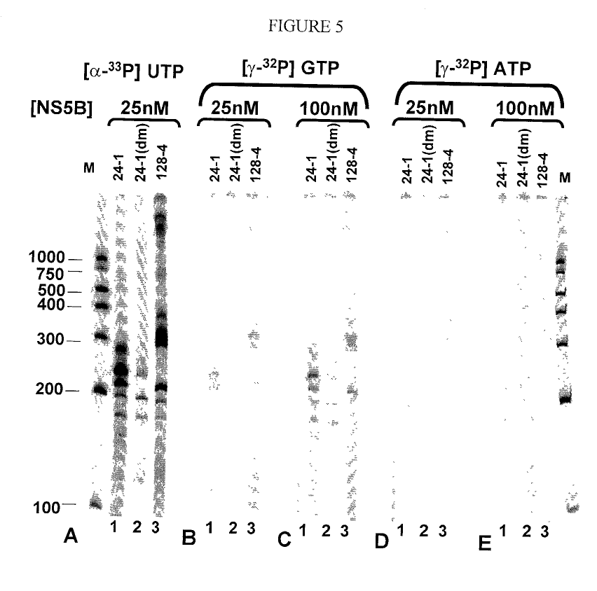

The characteristic products of NS5B primer-independent RNA synthesis from HCV 3′UTR templates are easily detected using [α-³³P]UTP precursors that incorporate multiple ³³P isotopes into the synthesized RNA backbone. Earlier studies have shown that the priming nucleotide incorporated into the RNA product by HCV NS5B maintains the 5′ tri-phosphate by the use of [γ-P³²]NTPs (Zhong et al., 2000). In order to further substantiate the role of template “CC” motifs as sites of de novo initiation by the HCV NS5B, we reconstituted the in vitro NS5B polymerase reaction with either [γ-P³²]GTP or [γ-P³²]ATP as the only radiolabeled tracer in the reaction. FIG. 5A displays the characteristic products (described above) that resulted from [α-P³³]UTP incorporation by 25 nM of NS5B polymerase with the 24-1 (lane 1), 24-1dm (lane 2), and 128-4 (lane 3) templates. FIG. 5B shows that the use [γ-P³²]GTP in the reaction as the radiolabel tracer, rather than [α-³³P]UTP, dramatically decreased the intensity of the products, supporting the proposal that only one mole of gamma-phosphate was incorporated per mole of RNA produced . Notwithstanding the decrease in product intensity, the products characteristic of initiation at template poly U/C “CC” motifs were detected with the [γ-P³²]GTP (FIG. 5 B) and were clearly evident from reactions catalyzed by higher (100 nM) concentrations of NS5B (FIG. 5C). The specificity of HCV NS5B in utilizing GTP as a primer at template “CC” motifs is highlighted by the lack of products detected with reactions that were reconstituted with [γ-P³²]ATP as the sole radioactive tracer (FIG. 5D and E).

### Example 5

### Evaluating the Effect of GTP Analogs on Primer-Independent RNA Synthesis by HCV NS5B

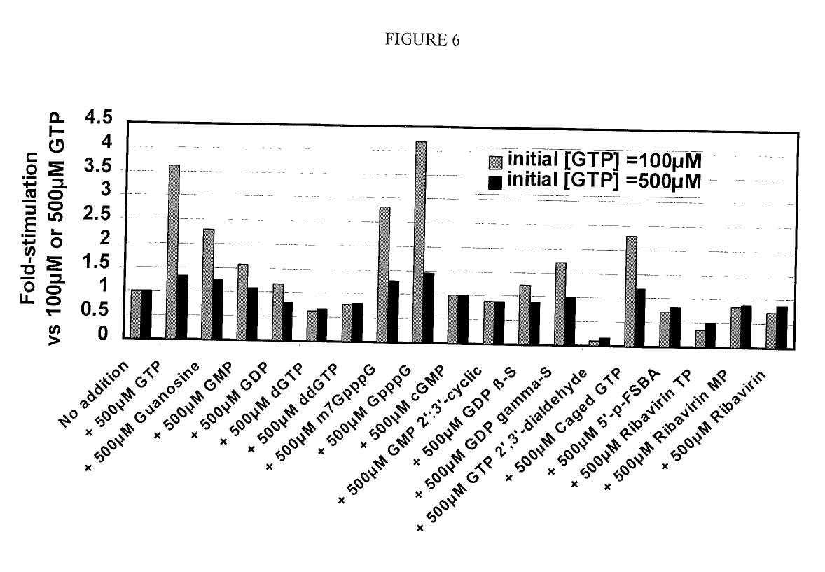

Primer-independent RNA synthesis by HCV NS5B is stimulated by high (0.5 to 1 mM) concentrations of either GTP or ATP (Lohmann et al., 1999; Oh et al., 1999; Luo et al., 2000; Zhong et al., 2000). The NS5B and HCV 3′-UTR templates described in this work, showed a similar activation by high concentrations of GTP. FIG. 6 depicts the results of two basic NS5B polymerase reactions reconstituted with the 24-1 template RNA. The hatched bars represent reactions primed with a basal level of 100 μM GTP in the reaction, and the closed bars represent reactions primed with a basal level of 500 μM GTP. Though the 100 μM GTP reaction produces less product, the fold-stimulation in product intensity (as measured by the incorporation of [α-³³P]UTP) for each of the two reference reactions is normalized to 1. Hence, the fold stimulation provided by a supplement of 500 μM GTP is greater in the reaction with 100 μM basal GTP, than in the reaction with 500 μM GTP, and reflects previously reported Km values for GTP in primer-independent RNA synthesis (Lohmann et al., 1999; Luo et al, 2000; Zhong et al., 2000).

A number of analogs were examined as potential substitutes for the stimulating GTP in the de novo initiation reaction with NS5B and the 24-1 RNA template. The di-nucleotide cap analogs GpppG and m7 GpppG were the most efficient analogs in stimulating de novo initiation, followed by guanosine, caged-GTP (fluorescently-tagged y-phosphate GTP), GMP and GDP-γ-S. The reactive GTP 2′3′-dialdehyde was a clear inhibitor. Moreover, the apparent consequence of supplementing 500 μM ribavirin tri-phosphate in the reference reactions was a moderate level of inhibition.

### Example 6

### Use of the HCV 3′UTR RNA Template 24-1 in A NS5B De Novo Initiation Reaction to Evaluate Inhibitors as Potential Anti-HCV Therapeutics

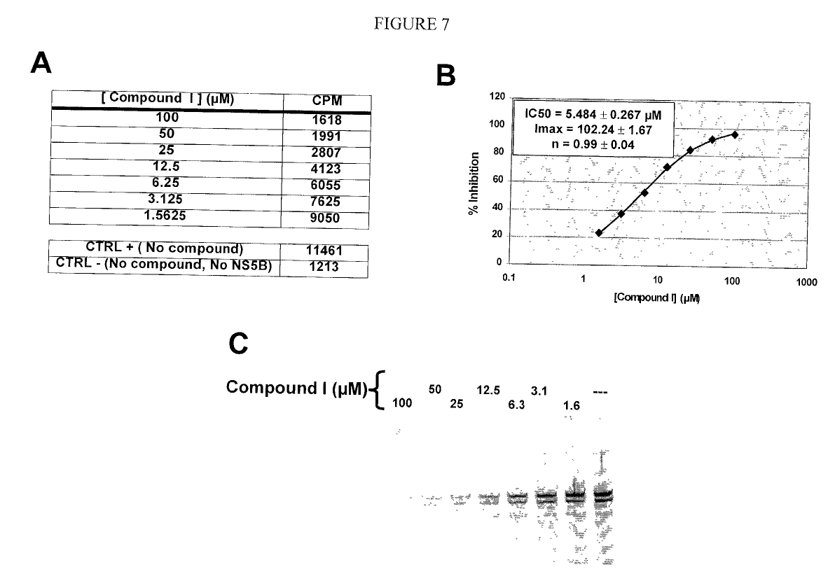

The assay presented in Example 1 was carried out for the purpose of screening potential inhibitors of the HCV NS5B polymerase and their effect on de novo initiation from the specific sites (described above) on the HCV 3′UTR RNA template 24-1. Compound I was added, at the indicated concentrations (FIG. 7), to the standard HCV polymerase reaction with template 24-1 as detailed in the materials and methods. For quantification purposes, 8μL of the reaction were spotted on a DE81 filter paper. After 3 washes (10 min each) in Na₂HPO₄/NaH₂PO₄ 1M pH 7, the filter was rinsed in water, then in EtOH and left to dry. Bound radioactivity, which is a measure of the amount of RNA produced in the primer-independent NS5B RNA polymerase reaction, was quantified by liquid scintillation counting (FIG. 7A).

The % inhibition was calculated with the following equation:

100−[(countᵢₙₕ-count_{blank})/(count_{ctl}-count_{blank})×100].

A non-linear curve fit with the Hill model was applied to the inhibition-concentration data, and the 50% effective concentration (IC₅₀) was calculated by the use of SAS software (Statistical Software System; SAS Institute, Inc. Cary, N.C.).

The % inhibition is plotted versus the compound concentration to obtain an IC₅₀ of 5.5 μM for compound I. Moreover, the specific inhibition of NS5B in the production of the 227 and 246 nt species (products stemming from internal de novo initiation) from template 24-1 demonstrate the utility of these compounds in evaluating specific inhibitors of HCV RNA transcription.

### Discussion

The examples of the present invention provide template-strand initiation sites comprising a polypyrimidine tract that are specifically recognized by RNA-dependent RNA polymerases, particularly the HCV NS5B polymerase. The precise initiation sites on the template strands are identified as cytidylate or poly-cytidylate clusters located in or adjacent to a polyuridylate tract. To date, there has been no explicit function associated with the poly U/U-C tract or a characterization for its role in the interaction with other HCV encoded activities. Though the poly U/U-C tract is a highly conserved segment of the HCV genome (Choo et al., 1989), the length of this segment varies, as exemplified by the cloning of different sized segments from the RNA genomes of an HCV-infected liver (Example 1). A feature of all these poly U/U-C tracts is the presence of short C or CC motifs that are located both at the 5′-end and within the long poly U stretches. This sequence organization is characteristic of all the genomic HCV RNAs examined. The examples of the present invention provide a specific role for the poly U/U-C tract and highlight the preference of the HCV NS5B polymerase for de novo initiation specifically at the template C motifs. This is the first evidence of a functional role for the 3′UTR poly U/U-C tract in HCV RNA-dependent RNA transcription and clearly demonstrates the requirement for the C motifs, thereby providing an explanation for the conservation of this distinctive organization among HCV RNA genomes.

Primer-independent, de novo initiation of RNA synthesis is a well characterized reaction of the HCV NS5B polymerase (Oh et al., 1999; Zhong et al, 2000; Luo et al., 2000; Sun et al., 2000), which is conserved among related flaviviral NS5B polymerases (Kao et al., 1999). The precise sites for de novo initiation are determined by the composition of the template sequence. NS5B RNA substrates that mimic the 3′-end of the HCV RNA negative strand, and terminate with a 3′-C, serve as templates to generate products that contain 5′ GTP as the initiating nucleotide on the product strand (Oh et al., 1999; Luo et al, 2000). In contrast, HCV NS5B RNA templates comprising the 3′-end of the HCV plus-strand generate different products. Oh et al. 2000 and Sun et al. 2000 demonstrate that RNA templates containing the poly U/U-C tract and the 3′-UTR region, are transcribed by the HCV NS5B into RNA products that are smaller than the unit length template. Oh et al., 2000 suggest that these products initiate at a site remote from the template 3′-end and prematurely terminate. Sun et al. 2000 and WO 2000/33635, published on 15 June 2000, disclose and claim initiation sequences specific for template-independent RNA synthesis catalyzed by HCV polymerase. They claim a distinct sequence in the HCV 3′-UTR template as a specific recognition site for de novo initiation by the HCV NS5B polymerase. Despite their observation that non-specific templates comprising a cluster of cytidylates also serve as substrate templates for HCV NS5B de novo initiation, the data presented explicitly argues that the HCV specific RNAs utilize a template uridylate as the initiation site. Some experimental data suggest that the polymerase has greater affinity for poly C than poly U. However, in the case of their experiments with HCV RNA templates, this data is contradicted by the complete lack of incorporation of [γ³²P]-GTP in resulting RNA products. They conclude that “the resulting nascent RNA products, smaller than the template used, contained ATP as the first nucleotide suggesting that a transcription initiation site in the HCV RNA genome is uridylate”. They explain the phenomenon of smaller RNA products on the basis of various stages in RNA synthesis which leads the reader to believe that the initiation observed is not carried out internally at different sites of the molecule but takes place at a single site:

“the major products of the reactions were about 370 nt in length i.e. about 100 bases smaller than the template. Meanwhile several minor RNA products, smaller than the template and the major RNA product, were present in the reaction, indicating various stages in RNA synthesis”. . .

They do not remotely suggest the possibility that the initiation sites may be scattered along the RNA, leading to RNA products of different length corresponding to the position of each initiation site. It is clear from the contradictory data, and lack of explanation thereof, that Sun et al. do not disclose the inventive concept of the present invention. Sun et al. were not in possession of the significance of the C cluster with a poly U tract as the specific site for de novo initiation of HCV NS5B polymerase. In addition, details lacking concerning the types of templates used (such as the specific sequence of their poly U/C template) provides no information whether their template contained a cluster of C within a poly U stretch or simply a stretch of U followed by a stretch of C. Even then, the data suggest that in a HCV template, U is the site of initiation and not C.

The examples of this invention unambiguously identify the C motifs of the HCV 3′-UTR poly U/UC tract as template directed initiation sites. The different lengths of the major run-off transcription products generated by the HCV NS5B from the templates labeled: 24, 128-4, 130-21 and 150-41 in Example 1, precisely correlate with the position of the C motifs in the poly U/U-C tract in the respective templates. Moreover, the subtle substitution of uridylates for specific cytidylates within one of the templates in Example 3, abolished the major products that are predicted to originate from the corresponding C motifs. Finally, the demonstration in Example 4 that a GTP constitutes the 5′-initiating nucleotide on the product RNA, as supported by the incorporation of a [γ³²P]-GTP, highlights a specific role for the poly U/U-C template cytidylate residues, in contrast to other claims (Sun et al., 2000. and WO 2000/33635; Zhong et al, 2000)

The distinction and utility of the present invention are both apparent in the subsequent evaluation of various compounds as agonists or antagonists of the GTP stimulated HCV NS5B de novo initiation reaction. Example 5 highlights compounds that are analogs of GTP, capable of either stimulating (such as the GpppG or 7-methyl GpppG) or inhibiting (such as GTP 2′3′-dialdehyde or ribavirin triphosphate) the NS5B reaction using these templates. Moreover, the sensitivity of the reaction to more potent inhibitors is evident from Example 6 wherein the NS5B catalyzed generation of the characteristic product using the HCV RNA template is specifically inhibited by a compound with an IC₅₀ in the low micromolar range. The in vitro reconstituted de novo initiation reaction is closer to the authentic mechanism of HCV RNA transcription and replication in vivo. The further discovery of de novo initiation sites and use in the establishment of an assay to measure a specific step in the NS5B RNA-dependent RNA polymerase reaction, disclosed herein, permitted the analysis of the initiation requirements. The utility of the RNA templates and sites that we have described are a significant part of the growing arsenal of tools used in the search for antiviral compounds specifically targeting the initial step of RNA synthesis by the HCV NS5B RNA polymerase.

What is claimed is:

1. An isolated de novo initiation site for an RNA-dependent RNA polymerase (RdRp), said site consisting essentially of a polypyrimidine tract and at least one cytidylate residue located therein, or adjacent thereto.

2. An isolated de novo initiation site for an RNA-dependent RNA polymerase, said site consisting essentially of a polypyrimidine tract comprising at least two or more adjacent cytidylate residues located therein, or adjacent thereto.

3. An isolated de novo initiation site for an RNA-dependent RNA polymerase, said site consisting essentially of a polypyrimidine tract comprising a cluster of cytidylate residues located therein, or adjacent thereto.

4. The de novo initiation site according to anyone of claims 1 to 3, wherein said RdRp is a flavivirus polymerase.

5. The de novo initiation site according to claim 4, wherein said polymerase is the Hepatitis C virus (HCV) NS5B RNA-dependent RNA polymerase, an analog, variant or derivative thereof.

6. The RNA initiation site according to claim 1, wherein said polypyrimidine tract essentially consists of greater than 90% of uridylate residues.

7. The RNA initiation site according to claim 1, comprising a sequence selected from the group consisting of: (P)ₙ(C)ₘ; (C)ₘ(P)ₙ; or (P)ₙ(C)ₘ(P)ₙ, wherein P is a pyrimidine or an analog thereof, each n is independently 2 to 200 and m is 1 to 10.

8. The RNA initiation site according to claim 7, wherein said P is polypyrimidine tract consisting essentially of greater than 90% uridylate residues.

9. The RNA molecule according to claim 1, comprising a CCC or CCCC sequence adjacent to a poly (U) tract.

10. An isolated de novo initiation site for an RNA-dependent RNA polymerase (RdRp), said site selected from the group consisting of:
U₁₃CU₃CCUUCU₃ (SEQ ID. NO. 21)
U₁₁GU₂₉CU₃₀CCU₄CCU₁₀AUAUUCCUUCU (SEQ ID. NO. 22);
U₁₄GGU₄₇CCU₅CCU₁₀AUAUUCCUUCU (SEQ ID. NO. 23); and
U₁₃GU₅₈CCU₁₃AUAUUCCUUCU (SEQ ID. NO. 24).

11. A RNA template for primer-independent RNA synthesis comprising the isolated de novo initiation site according to any one of claims 1 to 3 and 10 and a further RNA portion suitable for providing a template for elongation of said polymerase.

12. A method of identifying a compound that inhibits primer-independent de novo RNA synthesis catalyzed by an RNA-dependent RNA polymerase (RdRp), comprising the steps of:
a) contacting a RNA template according to claim 11, with said NS5B polymerase in the absence of a primer and in the absence of said compound under conditions permitting RNA synthesis, and determining the amount of RNA thus formed;
b) contacting a RNA template as in a), with said RdRp in the absence of a primer and in the presence of said compound under the same conditions as in a), and determining the amount of RNA thus formed; and
c) comparing the amount of RNA product formed in b) with that of a);
wherein any reduction in the amount of RNA product formed in b) compared with that formed in a) indicates a compound that is an inhibitor of primer-independent de novo RNA synthesis catalyzed by said polymerase.

13. The method according to claim 12, wherein said polymerase is a flavivirus RdRp.

14. The method according to claim 12, wherein said polymerase is the Hepatitis C virus NS5B polymerase.

15. The method according to claim 12, wherein said compound inhibits binding of said NS5B polymerase to said initiation site of said template.

16. The method according to claim 12, wherein said compound inhibits priming of said NS5B polymerase once bound to said initiation site of said template.

17. The method according to claim 12, wherein said compound inhibits elongation by said RNA polymerase along said further RNA portion of said template.

18. A method of inhibiting primer- independent de novo RNA synthesis catalyzed by an RNA-dependent RNA polymerase (RdRp) comprising the step of:
contacting said polymerase with a polymerase-inhibiting effective amount of a compound that inhibits primer-independent de novo RNA synthesis catalyzed by said polymerase,
wherein said compound is identified by the method according to claim 12.

19. The method according to claim 18, wherein said polymerase is a flavivirus RdRp.

20. The method according to claim 18, wherein said polymerase is the Hepatitis C virus NS5B polymerase.

21. The method according to claim 18, wherein said method provides inhibition of binding of said NS5B polymerase to said initiation site of said template.

22. The method according to claim 18, wherein said method provides inhibition of priming of said NS5B polymerase once bound to said initiation site of said template.

23. The method according to claim 18, wherein said method provides inhibition of elongation by said RNA polymerase along said further RNA portion of said template.

24. A method of inhibiting the replication of hepatitis C virus comprising the step of:
a) contacting said hepatitis C virus with an antiviral effective amount of a compound that inhibits primer-independent de novo RNA synthesis catalyzed by HCV NS5B polymerase,
wherein said compound is identified by the method according to claim 12.

25. The method according to claim 24, wherein said method provides inhibition of binding of said NS5B polymerase to said initiation site of said template.

26. The method according to claim 24, wherein said method provides inhibition of priming of said NS5B polymerase once bound to said initiation site of said template.

27. The method according to claim 24, wherein said method provides inhibition of elongation by said RNA polymerase along said further RNA portion of said template.

28. A method for producing a anti-HCV compound comprising the step of:
identifying said compound according to the method of claim 12.

29. A method of identifying a compound that inhibits primer-independent de novo RNA synthesis catalyzed by a flavivirus RNA-dependent RNA polymerase, comprising:
a) contacting a synthetic heteropolymeric RNA template comprising a cluster of cytidylate nucleotides or a mixed cluster of cytidylate and uridylate nucleotides with said polymerase in the absence of a primer and in the absence of said compound under conditions permitting RNA synthesis, and determining the amount of RNA product thus formed;
b) contacting an RNA template as in a) with said polymerase in the absence of a primer and in the presence of said compound under the same conditions as in a), and determining the amount of RNA product thus formed; and
c) comparing the amount of RNA product formed in b) with that in a),
wherein any reduction in the amount of RNA product formed in b) compared with that formed in a) indicates a compound that is an inhibitor of primer-independent de novo RNA synthesis catalyzed by said polymerase.

30. The method according to claim 24, wherein said polymerase is the Hepatitis C virus NS5B RNA-dependent RNA polymerase, an analog, variant or derivative thereof.
